SEQUENCE LISTING (4 sequences)

SEQ ID NO: 1 - protein, 261 aa, Homo sapiens
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 2 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 3 - protein, 20 aa, Artificial
  Synthetic peptide based on residues 141 to 160 of p21
KRRQTSMTDFYHSKRRLIFS

SEQ ID NO: 4 - protein, 25 aa, Artificial
  Synthetic peptide based on residues 141 to 160 of p21 fused to the RYIRS tag
KRRQTSMTDFYHSKRRLIFSRYIRS

(19) United States
(12) Patent Application Publication
(10) Pub. No.: US 2013/0190251 A1
(43) Pub. Date: Jul. 25, 2013

(54) METHOD FOR PREDICTING THE RESPONSIVENESS TO CHEMOTHERAPY

(71) Applicants: Veronique Witko-Sarsat, Paris (FR); Didier Bouscary, Paris (FR); Magali Pederzoli-Ribeil, Paris (FR); Olivier Hermine, Paris (FR); Isabelle Dusanter-Fourt, Paris (FR)

(72) Inventors: Veronique Witko-Sarsat, Paris (FR); Didier Bouscary, Paris (FR); Magali Pederzoli-Ribeil, Paris (FR); Olivier Hermine, Paris (FR); Isabelle Dusanter-Fourt, Paris (FR)

(21) Appl. No.: 13/635,448

(22) PCT Filed: Mar. 18, 2011
(86) PCT No.: PCT/EP11/54158

§ 371 Date: Nov. 26, 2012

(30) Foreign Application Priority Data
Mar. 18, 2010 (EP) 10305269.2

(51) Int. Cl. G01N 33/68; A61K 31/704; A61K 31/506; A61K 31/713; A61K 38/10

(52) U.S. Cl. CPC G01N 33/6893; A61K 31/713; A61K 38/10; A61K 31/506; A61K 31/704
USPC 514/19.6; 435/7.1; 514/44 A; 544/295; 536/6.4; 514/34

(57) ABSTRACT

The present invention concerns a method for predicting the responsiveness of an individual suffering from leukemia to a chemotherapeutic drug. In particular, this method comprises determining the proportion of leukemic cells expressing cytoplasmic PCNA in a biological sample of the individual. The present invention also relates to a tyrosine kinase inhibitor for use for the treatment of an individual suffering from leukemia and having a proportion of leukemic cells expressing cytoplasmic PCNA in a biological sample lower than a predetermined threshold. The invention also pertains to a method for diagnosing whether an individual suffers, or is at risk of suffering, from leukemia.

The present invention concerns a method for predicting the responsiveness of an individual suffering from leukemia to a chemotherapeutic drug. In particular, this method comprises determining the proportion of leukemic cells expressing cytoplasmic PCNA in a biological sample of the individual. The present invention also relates to a tyrosine kinase inhibitor for use for the treatment of an individual suffering from leukemia and having a proportion of leukemic cells expressing cytoplasmic PCNA in a biological sample lower than a predetermined threshold. The invention also pertains to a method for diagnosing whether an individual suffers, or is at risk of suffering, from leukemia.

Acute Myelocytic Leukemia (AML)

In acute myelocytic leukemia (AML), an accumulation of immature blastic precursors blocked at a more or less advanced stage of differentiation can be observed. These cells do not differentiate and are resistant to apoptosis. The prognostic for a patient suffering from AML is rather severe because, for the forms of AML associated with a normal karyotype, the probability of success of the available unspecific therapies (e.g. anthracyclin and cytarabine) is only of 50%. It was demonstrated that, during AML, the leukemic cells have a proliferative advantage and a defect in one or several apoptotic pathways. This defect in apoptosis is frequently associated with resistance to treatment and to low rates of survival of the patients.

To date, no prognostic marker of resistance to apoptosis-triggering treatments exists for patients suffering from AML and the prognostic remains severe.

Chronic Myelocytic Leukemia (CML)

The chronic myelocytic leukemias (CML) are slow-progressing cancers characterized by a proliferation of circulating cells belonging to the myeloid lineage. They are characterized by the presence of the Philadelphia chromosome and the expression of the Bcr-Abl fusion protein, which has a constitutive intrinsic kinase activity. Imatinib mesilate (commercially available as Gleevec®) specifically targets this kinase and is successfully used as a drug. However, resistance to imatinib mesilate treatment may occur in 5% to 10% of the cases. In addition, this treatment can only cure less than 10% of the patients because removal of the treatment leads to relapse. The molecular mechanism underlying this resistance is not known but might be related to intrinsic properties of a subpopulation of leukemic stem cells.

Therefore, there is a need to find prognostic markers of resistance to imatinib mesilate treatment for patients suffering from CML.

PCNA and Apoptosis Regulation

PCNA has long been believed to be a nuclear protein, having a crucial role in DNA replication and repair in proliferating cells only.

Some studies have demonstrated that a higher expression of PCNA in myeloblastic cells correlates with blast accumulation in myelodysplasic syndrome (Kracmarova et al. Leuk Lymphoma 2008; 49:1297-1305) and in relapsed acute myelocytic leukemia (Staber et al. Oncogene 2004; 23:894-904). In chronic myelocytic leukemia, PCNA has been shown to be up-regulated (Bruchova et al. Leuk Lymphoma 2002; 43:1289-1295), and it was demonstrated that silencing PCNA expression by small interfering RNA leads to increased apoptosis and decreased proliferation of leukemic cells (Merkerova et al. Leuk Res. 2007; 31:661-672).

However, the potential use of PCNA as a marker of responsiveness to anti-leukemia treatment has not been investigated. In addition, the cellular localization of PCNA in cells from patients suffering from leukemia has not been investigated, either.

The inventors have recently shown that in neutrophils, PCNA localizes exclusively in the cytoplasm, due to a relocalization occurring during myeloid differentiation. Moreover, they have shown that in neutrophils, cytosolic PCNA levels change in parallel with cellular survival rate. More specifically, cytosolic PCNA levels decrease during apoptosis and increase during in vitro or in vivo exposure to the survival factor G-CSF. Therefore, cytoplasmic PCNA acts as a cell cycle-independent regulator of neutrophil lifespan. In addition, they have shown that in a healthy person, PCNA is nuclear before granulocytic differentiation in myeloid precursors, and becomes exclusively cytoplasmic at the end of differentiation in mature neutrophils. Moreover, the nucleo-cytoplasmic transport depends on a sequence of nuclear export.

The inventors have now unexpectedly found that cytoplasmic PCNA can be detected in promyelocytes isolated from the bone marrow of patients suffering from myelocytic leukemia, in contrast to what is observed in bone marrow promyelocytes isolated from a healthy subject. In other terms, an aberrant localization of PCNA is observed in patients suffering from AML.

More specifically, in myeloid leukemic cell lines (e.g. UT7, HL60), PCNA is expressed in the cytoplasm. Furthermore, the level of cytoplasmic PCNA is increased in myeloid leukemic cell lines resistant to treatments (e.g. UT7 resistant to doxorubicine) in comparison with susceptible cell lines. In cells isolated from patients suffering from AML, some myeloid precursors display cytoplasmic PCNA, whereas precursors from normal bone marrow only bear nuclear PCNA.

The K562 cell line is derived from a CML and is characterized by the presence of the Philadelphia chromosome and by the expression of the Bcr-Abl fusion protein. The inventors have shown that the expression of cytoplasmic PCNA was increased in this cell line, which is resistant to doxorubicin and imatinib mesilate. A similar observation has been made in the myeloid cell line UT7-9 stably transfected with Bcr-Abl. Therefore, PCNA can be considered as a prognostic marker of resistance to imatinib mesilate in CML.

The inventors have thus provided evidence that cytoplasmic PCNA is associated with a decreased susceptibility to apoptosis and increased drug resistance in myeloid leukemic cells. Therefore, in the case of AML, cytoplasmic PCNA can be considered as a prognostic marker of the responsiveness to treatment that may help in making a decision towards a transplant. If the patient is likely to respond to a treatment by chemotherapy (low levels of cytoplasmic PCNA), then such treatment may be administrated to him/her. To the contrary, if the patient is likely not to respond to a treatment by chemotherapy (high levels of cytoplasmic PCNA), then the patient may need a transplant.

Method for Predicting the Responsiveness of a Patient to a Chemotherapeutic Drug

The present invention pertains to a method for predicting the responsiveness of an individual suffering from leukemia to a chemotherapeutic drug, said method comprising determining the proportion and/or percentage of leukemic cells expressing cytoplasmic PCNA in a biological sample of the individual.

As used throughout the present specification, the term “PCNA” refers to the human Proliferating Cell Nuclear Antigen protein. In a preferred embodiment, “PCNA” refers to a protein of sequence SEQ ID NO: 1. However, this term also encompasses allelic variants and splice variants of the protein of SEQ ID NO: 1. In the frame of the present invention, the PCNA protein is a cytoplasmic PCNA, most preferably a cytoplasmic PCNA found in myeloblasts or promyelocytes.

As used herein, the term “leukemia” refers to a cancer of the white blood cells involving bone marrow, circulating white blood cells, and organs such as the spleen and lymph nodes. As used herein, this term both encompasses acute leukemia (which consist of predominantly immature, poorly differentiated cells—usually blast forms), chronic leukemia (which involve more mature cells), and the myelodysplastic syndrome.

Leukemia can be subdivided into two groups, according to which kind of blood cell is affected. Lymphocytic leukemia involves leukemic cells belonging to the lymphoid lineage, whereas myelocytic leukemia involves leukemic cells belonging to the myeloid lineage. Preferably, the leukemia according to the invention is a myelocytic leukemia, e.g. an acute myelocytic leukemia or a chronic myelocytic leukemia.

In leukemia, white blood cells (also called leukocytes) display unregulated growth and proliferation and lack of differentiation, due to loss of normal controls. Such cells are referred to as “leukemic cells”. Leukemic cells are precursor cells of the myeloid lineage (which differentiate in granulocytes, monocytes and dendritic cells) such as myeloblasts, promyelocytes and promonocytes, or precursor cells of the lymphoid lineage (which differentiate in lymphocytes, natural killers and dendritic cells) such as lymphoblasts and prolymphocytes. Preferably, the expression “leukemic cells” refers to myeloblasts or promyelocytes.

Leukemia may be treated with different kinds of treatments well-known by the skilled in the art, among which chemotherapy. Chemotherapy is a treatment based on the use of biochemical agents (e.g. chemical molecules, antibodies, polypeptides, polynucleotides, etc.), which are referred to as “chemotherapeutic drugs”. Such chemotherapeutic drugs used for the treatment of cancers, such as for instance for the treatment of leukemia, include for example antineoplastic drugs selected from the group consisting of an alkaloid, an alkylating agent, an antimetabolite (e.g. a nucleoside analog), an antibiotic, a tyrosine kinase inhibitor, a topoisomerase inhibitor, a monoclonal antibody, a biological response modifier (IFN) and a corticosteroid.

In particular, chemotherapeutic drugs commonly used for the treatment of myelocytic leukemia include alkaloids such as vincristine, alkylating agents such as busulfan, antimetabolites such as cytarabine, 6-thioguanine and hydroxyurea, antibiotics such as daunorubicin and idarubicin, tyrosine kinase inhibitors such as imatinib mesilate, topoisomerase inhibitors such as etoposide and doxorubicin, monoclonal antibodies such as gemtuzumab ozogamicin and biological response modifiers such as IFN-alpha.

The drugs used in anti-leukemia chemotherapy may vary according to the type of leukemia. In particular, chemotherapeutic drugs used in the case of acute myelocytic leukemia include e.g. cytarabine, daunorubicin, idarubicin, 6-thioguanine, etoposide as induction therapy, and gemtuzumab ozogamicin (a recombinant monoclonal antibody combined with a cytotoxic drug) in the case of relapse.

In contrast to this, chemotherapeutic drugs used in the case of chronic myelocytic leukemia include e.g. imatinib mesilate as a first-line treatment, and other kinase inhibitors such as dasatinib and nilotinib in the case of ABL-BCR-negative patients, of patients who relapse after receiving imatinib mesilate, and/or of patients in blast crisis. In any case, these treatments might be followed by allogenous bone marrow transplantation.

In a preferred embodiment, the chemotherapeutic drug is a tyrosine kinase inhibitor or a topoisomerase inhibitor. By “tyrosine kinase inhibitor” is meant a molecule able to prevent the biological activity of an enzyme that transfers a phosphate group from ATP to a tyrosine residue in a protein. By “topoisomerase inhibitor” is meant a molecule able to prevent the biological activity of an enzyme that control the changes in DNA structure by catalyzing the breaking and rejoining of the phosphodiester backbone of DNA strands.

In another preferred embodiment, the chemotherapeutic drug of the present invention is imatinib mesilate (commercially available as Gleevec® or Glivec®) or doxorubicin (commercially available as Myocet®).

As shown in example 3, the presence of a high amount of cytoplasmic PCNA is associated with drug resistance in myeloid leukemic cells. Thus, cytoplasmic PCNA can be used as a marker for predicting the responsiveness of a patient to a drug.

By “predicting the responsiveness” of a patient to a drug is meant evaluating the chance of resolution or improvement of abnormal clinical features. For example, in a patient suffering from leukaemia that responds to a drug, a restoration of normal blood counts and of normal hematopoiesis (e.g. with <5% blast cells) can be observed and the leukemic clone(s) can be eliminated when the patient response respond to the drug. More specifically, “predicting the responsiveness” of a patient to a drug includes predicting whether upon a treatment with said drug, the patient is likely to undergo a complete remission, a partial remission, a remission with a high or a low risk of relapse, or whether said treatment will have no significant effect on the abnormal clinical features and/or the evolution of the disease.

More precisely, the present invention relates to a method based on the determination of the proportion of leukemic cells expressing cytoplasmic PCNA in a biological sample of an individual. By “determining the proportion” of leukemic cells expressing cytoplasmic PCNA is meant counting the number of leukemic cells expressing cytoplasmic PCNA and the number of leukemic cells expressing exclusively nuclear PCNA and calculating the ratio of leukemic cells expressing cytoplasmic PCNA to total leukemic cells. Counting the number of leukemic cells expressing cytoplasmic PCNA may be performed by various methods well-known by one skilled in the art. For instance, it can be performed by immunocytochemistry (see Example 1), by western blot, or by flow cytometry (FACS).

In a preferred embodiment, a proportion of leukemic cells expressing cytoplasmic PCNA higher than a predetermined threshold indicates that the individual is likely not to respond to the chemotherapeutic drug. The term “predetermined threshold” refers to the mean proportion of leukemic cells expressing cytoplasmic PCNA in a biological sample of a leukemia-suffering individuals who display a good response to the chemotherapeutic drug.

More preferably, a proportion of leukemic cells expressing cytoplasmic PCNA of at least 40%, 45%, 50%, 55% or 60% is indicative that the individual is likely not to respond to the chemotherapeutic drug. More preferably, a proportion of leukemic cells expressing cytoplasmic PCNA of at least 50% is indicative that the individual is likely not to respond to the chemotherapeutic drug. Still more preferably, a proportion of leukemic cells expressing cytoplasmic PCNA of at least 70%, 80% or 90% is indicative that the individual is likely not to respond to the chemotherapeutic drug.

Depending on the age of the patient, on the diagnosis and on the stage of the disease, a patient suffering from leukemia may be treated by different means. Most of the time, the primary treatment for leukemia involves chemotherapy. However, bone marrow transplantations may alternatively be performed, possibly in combination with high-dose chemotherapy and/or radiation. Bone marrow transplant may for instance be needed when a more advanced, or uncontrolled state of the disease is reached, or when the patient do not respond to chemotherapy or cannot tolerate chemotherapy. However, bone marrow transplant remains harmful, as the patient may die from this procedure, and requires finding a compatible donor. Therefore, in general, bone marrow transplant is only performed when the patient does not respond to chemotherapy.

The method according to the invention allows predicting the responsiveness of a patient to chemotherapy and thus helps in designing a treatment regimen. In particular, if the patient is unlikely to respond to chemotherapy, it is advisable to directly opt for bone marrow transplantation (optionally in combination with high-dose chemotherapy and/or radiation).

Thus, in a preferred embodiment, the method of the present invention further comprises a step of designing a treatment regimen for the patient.

In the present application, the expression “treatment regimen” refers to the kind of therapeutical means used to treat a patient. The treatment regimen of a patient suffering from leukemia may for instance include chemotherapy, biological therapy, radiation therapy, or bone marrow transplantation, performed alone or in combination. Preferably, the treatment regimen of a patient having a proportion of leukemic cells expressing cytoplasmic PCNA lower than a predetermined threshold should include chemotherapy. Also preferably, the treatment regimen of a patient having a proportion of leukemic cells expressing cytoplasmic PCNA higher than a predetermined threshold should include means of treatment other than chemotherapy, alone or in combination with chemotherapy. Such means of treatment may for instance include biological therapy, radiation therapy and/or bone marrow transplant.

The method of the present invention may apply to any biological sample. The term “biological sample” refers to any type of biological sample containing leukemic cells. The biological sample may e.g. correspond to leukemic cells obtained from a biological fluid such as blood. The biological sample most preferably corresponds to blood. The biological fluid may optionally be enriched for leukemic cells, or leukemic cells may optionally be isolated from biological fluid. Enrichment for or isolation of leukemic cells may be achieved using, for example, flow cytometry (FACS) with an antibody directed to a leukemic cell-specific antigen, or using magnetic beads or other solid supports (for example a column) coated with an antibody directed to a leukemic cell-specific antigen.

In the frame of the present invention the individual is a mammal, preferably a human being.

Compounds for the Treatment of an Individual Suffering from Leukemia

As shown in example 3, cytoplasmic PCNA is associated with resistance of myelocytic leukemia cell lines to different chemotherapeutic drugs. Thus, the invention further pertains to a chemotherapeutic drug for use for the treatment of an individual suffering from leukemia, said individual having a proportion of leukemic cells expressing cytoplasmic PCNA in a biological sample lower than a predetermined threshold.

The term “treatment” is understood to mean treatment for a curative purpose (aimed at alleviating or stopping the development of the pathology) or for a prophylactic purpose (aimed at reducing the risk of appearance of the pathology).

The chemotherapeutic drug of the invention can correspond to any one of the chemotherapeutic drugs described hereabove in the paragraph entitled “Method for predicting the responsiveness of a patient to a chemotherapeutic drug”. In a preferred embodiment, the chemotherapeutic drug is a tyrosine kinase inhibitor or a topoisomerase inhibitor. Most preferably, it is imatinib mesilate or doxorubicin.

The chemotherapeutic drug of the present invention may be administered by any route that achieves the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use. Parenteral route is particularly preferred.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Depending on the intended route of delivery, the chemotherapeutic drug may be formulated as liquid (e.g., solutions, suspensions), solid (e.g., pills, tablets, suppositories) or semisolid (e.g., creams, gels) forms.

In a preferred embodiment, the individual to be treated with the chemotherapeutic drug suffers from myelocytic leukemia. More preferably, the individual to be treated with the chemotherapeutic drug suffers from acute myelocytic leukemia or chronic myelocytic leukemia.

The invention also pertains to a method for treating a leukemia comprising the step of administering an effective amount of a chemotherapeutic drug as defined herein to an individual having a proportion of leukemic cells expressing cytoplasmic PCNA in a biological sample that is lower than a predetermined threshold.

By “effective amount” is meant an amount sufficient to achieve a concentration of chemotherapeutic drug which is capable of preventing, treating or slowing down the disease to be treated. Such concentrations can be routinely determined by those of skilled in the art. The amount of the compound actually administered will typically be determined by a physician or a veterinarian, in the light of the relevant circumstances, including the condition to be treated, the chosen route of administration, the actual compound administered, the age, weight, and response of the subject, the severity of the subject's symptoms, and the like. It will also be appreciated by those of skilled in the art that the dosage may be dependent on the stability of the administered drug.

According to the present invention, the individual to be treated with the chemotherapeutic drug has a proportion of leukemic cells expressing cytoplasmic PCNA lower than a predetermined threshold. The term “predetermined threshold” refers to the mean proportion of leukemic cells expressing cytoplasmic PCNA in a biological sample of leukemia-suffering patients who display a good response to the chemotherapeutic drug.

In a preferred embodiment, the individual to be treated with the chemotherapeutic drug has a proportion of leukemic cells expressing cytoplasmic PCNA of at most 60%, 55%, 50%, 45% or 40%. More preferably, the individual to be treated with the chemotherapeutic drug has a proportion of leukemic cells expressing cytoplasmic PCNA of at most 30%, 20% or 10%.

Diagnosis of Leukemia

The inventors have shown that cytoplasmic PCNA can be detected in promyelocytes isolated from the bone marrow of patients suffering from myelocytic leukemia, in contrast to what is observed in bone marrow promyelocytes isolated from a healthy subject.

Thus, another aspect of the present invention pertains to a method for diagnosing whether an individual suffers from leukemia, said method comprising determining whether precursor cells of the myeloid or lymphoid lineage express cytoplasmic PCNA in a biological sample of the individual. In this method, the detection of cytoplasmic PCNA in precursor cells of the myeloid or lymphoid lineage indicates that the individual suffers from leukemia.

In a preferred embodiment, the above method is used for diagnosing a myelocytic leukemia. In this embodiment, it is determined whether precursor cells of the myeloid lineage, preferably promyelocytes, express cytoplasmic PCNA.

Counting the number of leukemic cells expressing cytoplasmic PCNA may be performed by various methods well-known by one skilled in the art, such as e.g. by immunochemistry, for example as described in Example 1.

Drug Monitoring and Monitoring of the Efficiency of a Treatment

The above method for predicting the responsiveness of an individual suffering from leukemia to a chemotherapeutic drug is also useful for monitoring progression of leukemia and/or for monitoring the efficiency of a treatment. In such a case, the method according to the invention described hereabove is repeated on another biological sample of the same patient at least at two different points in time. The biological samples may for example have been taken before and after beginning of an anti-leukemia treatment, respectively.

The invention therefore provides a method of monitoring the progression of leukemia comprising the steps consisting of:
a) determining the proportion and/or percentage of leukemic cells expressing cytoplasmic PCNA in a biological sample of an individual; and
b) repeating step a) on another biological sample of the same individual, taken at a later point in time;
   wherein a decrease in the proportion and/or percentage of leukemic cells expressing cytoplasmic PCNA in the course of time is indicative of an improvement of said individual's condition.

In particular, said method may comprise determining the proportion and/or percentage of leukemic cells expressing cytoplasmic PCNA in a patient who is undergoing a treatment, wherein a decrease in said proportion and/or percentage in the course of multiple myeloma treatment is indicative of an efficient treatment.

Accordingly, the invention also relates to a method of monitoring efficiency of a treatment of leukemia comprising the steps consisting of:
a) determining the proportion and/or percentage of leukemic cells expressing cytoplasmic PCNA in a biological sample of an individual before onset of said treatment; and
b) determining the proportion and/or percentage of leukemic cells expressing cytoplasmic PCNA in a biological sample of said individual after onset of said treatment;
   wherein a decrease in the proportion and/or percentage of leukemic cells expressing cytoplasmic PCNA in the course of time indicates that said treatment is efficient for treating said individual.

The monitoring of disease progression or treatment efficiency is typically performed by determining the number of genes expressed at different points in time, for instance at 2-week, 1-month, 2-month, 3-month intervals, etc.

A “decrease in the proportion and/or percentage of leukemic cells expressing cytoplasmic PCNA” is evaluated by comparing the proportion and/or percentage of leukemic cells expressing cytoplasmic PCNA when monitoring is started with the proportion and/or percentage of leukemic cells expressing cytoplasmic PCNA at any point in time. Said decrease is preferably statistically significant. A statistically significant decrease can for example correspond to a decrease of at least 5, 10, 25 or 50%.

Method for Selecting Patients to be Treated by a Combination of a Chemotherapeutic Drug and an Antagonist of Cytoplasmic PCNA

The above methods for predicting the responsiveness of a patient may also be used for designing a treatment regimen.

When the above methods are used to design a treatment regimen, they further comprise the step of designing a treatment regimen based on the proportion of leukemic cells expressing cytoplasmic PCNA in the biological sample of the individual.

Typically, the patient is given a treatment regimen comprising a combination of a chemotherapeutic drug and an antagonist of cytoplasmic PCNA if the proportion of leukemic cells expressing cytoplasmic PCNA is higher than a predetermined threshold, said threshold being indicative that the individual is likely not to respond to a chemotherapeutic drug alone. On the other hand, if the proportion of leukemic cells expressing cytoplasmic PCNA is lower than said predetermined threshold, said individual may be given the chemotherapeutic drug alone since it is likely to respond to the chemotherapeutic treatment.

More generally, patients who display a high expression level of PCNA in the cytoplasm of their cells need to be treated by a therapy comprising a combination of chemotherapeutic drug and of an antagonist of cytoplasmic PCNA. Cytoplasmic PCNA can thus be used as a marker for selecting the treatment regimen of a patient.

The invention is thus directed to an in vitro method for selecting a patient suffering from leukemia suitable to be treated with a therapy comprising a combination of a chemotherapeutic drug and an antagonist of cytoplasmic PCNA, said method comprising the steps of:

a) providing or obtaining a biological sample comprising leukemic cells;

b) determining the proportion of leukemic cells expressing cytoplasmic PCNA in said biological sample; and

c) selecting the patient having a proportion of leukemic cells expressing cytoplasmic PCNA higher than a predetermined threshold.

The invention is also directed to an in vitro method for selecting a patient suffering from leukemia suitable to be treated with a therapy comprising a chemotherapeutic drug, said method comprising the steps of:

a) providing or obtaining a biological sample comprising leukemic cells;

b) determining the proportion of leukemic cells expressing cytoplasmic PCNA in said biological sample; and

c) selecting the patient having a proportion of leukemic cells expressing cytoplasmic

PCNA lower than a predetermined threshold.

In a specific embodiment, the predetermined threshold is equal to 40%, and more preferably to 50%, 55%, 60%, 70%, 80% or 90%. Most preferably, said threshold is of 50%.

The chemotherapeutic drug is any of the chemotherapeutic drugs defined hereabove. In a specific embodiment, the chemotherapeutic drug is a tyrosine kinase inhibitor or a topoisomerase inhibitor. In another preferred embodiment, the chemotherapeutic drug of the present invention is imatinib mesilate (commercially available as Gleevec® or Glivec®) or doxorubicin (commercially available as Myocet®).

Combination of a Chemotherapeutic Drug and an Antagonist of Cytoplasmic PCNA for Use for the Treatment of an Individual Suffering from Leukemia

The inventors have provided evidence that cytoplasmic PCNA is associated with a decreased susceptibility to apoptosis and increased drug resistance in myeloid leukemic cells (see Example 3). Besides, the inventors have also shown that antagonists of cytoplasmic PCNA can sensitize daunorubicin-resistant cells to apoptosis (see Examples 5 and 6).

Therefore the invention also pertains to a combination of a chemotherapeutic drug and of an antagonist of cytoplasmic PCNA for use for the treatment of an individual suffering from leukemia, said individual having a proportion of leukemic cells expressing cytoplasmic PCNA higher than a predetermined threshold, and to the use of an antagonist of cytoplasmic PCNA for use for sensitizing cells to apoptosis in a patient suffering from leukemia, said individual having a proportion of leukemic cells expressing cytoplasmic PCNA in a biological sample higher than a predetermined threshold.

In a specific embodiment, the individual to be treated with a combination of a chemotherapeutic drug and an antagonist of cytoplasmic PCNA has a proportion of leukemic cells expressing cytoplasmic DNA of at least 40%, 50%, 55%, 60%, 70%, 80% or 90%, most preferably of at least 50%.

The chemotherapeutic drug is any of the chemotherapeutic drugs defined hereabove. In a specific embodiment, the chemotherapeutic drug is a tyrosine kinase inhibitor or a topoisomerase inhibitor. In another preferred embodiment, the chemotherapeutic drug of the present invention is imatinib mesilate (commercially available as Gleevec® or Glivec®) or doxorubicin (commercially available as Myocet®).

The “antagonist of cytoplasmic PCNA” according to the invention can for instance reduce the expression of cytoplasmic PCNA or inhibit cytoplasmic PCNA biological activity. The “antagonist of cytoplasmic PCNA” may for example correspond to a peptide, a small molecule, a nucleic acid (e.g. an antisense molecule, a shRNA or a siRNA), an antibody or an aptamer.

In a specific embodiment, said “antagonist of cytoplasmic PCNA” is a compound inhibiting an interaction between Proliferating Cell Nuclear Antigen (PCNA) and at least one polypeptide liable to bind to PCNA, as defined in PCT application PCT/EP2011/052760.

In a specific embodiment, the antagonist of cytoplasmic PCNA is a peptide. The peptide according to the invention can for example correspond to a fragment of at least 6, 10, 15 or 20 consecutive amino acids of PCNA or of a polypeptide liable to bind to PCNA such as e.g. p21, DNA polymerases, Clamp loader (Rfc1, Rfc3), Flpa-endonuclease (FEN-1), DNA ligase-1, topoisomerase II alpha, replication licensing factor (Cdt1), helicases and ATPases (Rrm3, WRN, RECQ5), mismatch repair enzymes (UNG2, MPG, hMYH, APE2), nucleotide excision repair enzyme (XPG), histone chaperone (CAF-1), poly(ADP-ribose) polymerase (PARP-1), chromatin remodelling factor (WSTF), DNA methyltransferase (DNMT1), sister-chromatid cohesion factors (Eco1, Chl1), cell cycle regulators (p57), and apoptosis regulators (ING1b, p53).

In a specific embodiment, the peptide can comprise or consist of a fragment of PCNA. Such a fragment preferably comprises at least 6, 10, 15 or 20 consecutive amino of the interdomain connecting loop of PCNA. Indeed, as shown in application PCT/EP2011/052760, peptides that comprise a PCNA fragment located in the interdomain connecting loop are capable of triggering neutrophil apoptosis.

Alternatively, the peptide can comprise or consist of a fragment of at least 6, 10, 15 or 20 consecutive amino acids of p21. Indeed, as shown in Example 6, the p21 peptide restores apoptosis in daunorubicin-resistant HL60. Such a fragment preferably comprises or consists of at least 6, 10, 15 or 20 consecutive amino acids of the p21 fragment spanning from residues 141 to 160 of p21, or comprises or consists of residue 141 to 160 of p21, optionally fused to a cell penetrating peptide such as, e.g., the peptide of sequence RYIRS. Indeed, as shown in PCT application PCT/EP2011/052760, the carboxyp21 peptide is capable of triggering neutrophil apoptosis. In a specific embodiment, the peptide comprises or consists of sequence SEQ ID NO: 3 (residues 141 to 160 of p21) or sequence SEQ ID NO: 4 (residues 141 to 160 of p21 fused to the RYIRS tag).

As used herein, the term “p21” refers to the human p21 protein, also called p21/Waf1/Cip1, CAP20, CDKN1, CIP1, MDA-6, p21CIP1, SDI1 or WAF1. In a preferred embodiment, “p21” refers to a protein of sequence SEQ ID NO: 2. However, this term also encompasses allelic variants and splice variants of the protein of SEQ ID NO: 2.

The peptide according to the invention may further comprise a tag, e.g. a tag enhancing entry of the peptide into cells.

In another embodiment, the antagonist of cytoplasmic PCNA is a acid nucleic, such as e.g. a siRNA or a shRNA targeting PCNA. Indeed, the inventors have shown that knocking down PCNA expression by siRNA sensitizes daunorubicin-resistant HL-60 cells to apoptosis.

In a preferred embodiment, the antagonist of cytoplasmic PCNA for use for the treatment of an individual suffering from leukemia induces apoptosis of leukemic cells. Determining whether a compound induces apoptosis of leukemic cells may be measured by various methods well-known by one skilled in the art. For instance, it may be quantified by measuring the amount of externalized phosphatidylserine, e.g. after annexin-V labeling. In such an experiment, externalized phosphatidylserine may be stained with a fluorochrome-coupled annexin V, thus allowing detection of apoptotic cells by flow cytometry.

All references cited herein, including journal articles or abstracts, published or unpublished patent application, issued patents or any other references, are entirely incorporated by reference herein, including all data, tables, figures and text presented in the cited references.

The invention will be further evaluated in view of the following examples and figures.

### BRIEF DESCRIPTION OF THE FIGURES

FIG. 1. PCNA expression in bone marrow cells of health donor and acute myelocytic leukemia (AML) donor. PCNA was detected by immunofluorescence using rabbit polyclonal anti-PCNA antibody. MPO positive cells (myeloid cells) were detected by immunofluorescence using mouse monoclonal anti-MPO antibody. The nuclear were visualized by staining of hoechst and the cells visualized by confocal scanning microscope.

FIG. 2. PCNA expression in myeloid leukemic cells line sensitive (K562S) or resistant (K562R) to doxorubicin. PCNA was detected by immunofluorescence using rabbit polyclonal anti-PCNA antibody. Nuclear and cytoplasmic fluorescence intensity in percentage of cells was quantified by Image J 1.42 software. The data are the mean±SEM of three independent experiments, p***<0.001 (student's t test).

FIG. 3. PCNA expression in myeloid leukemic cells line sensitive (UT7/9 S) or resistant (UT7/9R) to imatinib mesilate. PCNA was detected by immunofluorescence using rabbit polyclonal anti-PCNA antibody. Nuclear and cytoplasmic fluorescence intensity in percentage of cells was quantified by Image J 1.42 software. The data are the mean±SEM of three independent experiments, p*<0.01 (student's t test).

FIG. 4. Acquisition of drug resistance of UT7/9 by imatinib mesilate. Analysis of cell death of sensitive UT7/9 cells (S) or imatinib mesilate resistant (R) UT7/9 cells by phosphatyldylserine externalization assessed by annexin V-FITC binding and 7-AAD incorporation by FACS.

FIG. 5. Acquisition of drug resistance of UT7/9 and K562 by imatinib mesilate and doxorubicin. Counting of survival cell number in sensitive and resistance cells at Day 0 (D0) and Day 2 (D2) cultures (sensitive or doxorubicin resistant K562 cells, and sensitive or imatinib mesilate resistant UT7/9 cells).

FIG. 6. Acquisition of drug resistance of myelocytic leukemia cell line K562 by doxorubicin. Analysis of inhibition by cisplatin of DIOC2 incorporation in myelocytic leukemia K562 cell line. Drug resistance is mediated by ABC transporters such as P-glycoprotein (P-gp) K562 cells were incubated with DIOC2 with or without cisplatin, and the fluorescence of DIOC2 incorporated in cells was assessed by FACS. In K562-doxorubicin sensitive cells, high fluorescence of DIOC2 is observed because of the lack of efflux. No effect of cisplatin is observed consistent with an absence of ABC transporter-mediated drug resistance. In K562-doxorubicin resistant cells, low fluorescence of DIOC2 is observed due to its efflux which was reversed by cisplatin, consistent with the presence of an active ABC transporter-mediated drug resistance.

FIG. 7. The p21 peptide restores apoptosis in daunorubicin-resistant HL60 cells. HL60S and HL60R were incubated overnight with the p21 peptide (50 mM) or with gliotoxin (1 mg/ml). After a 15-hour incubation, the effect of the p21 peptide on the percentage of apoptotic HL60 cells was measured by mitochondrial depolarization after DiOC₆ labeling and by DNA fragmentation after propidium iodide labeling. Data are means±SEM of 5 independent experiments. Gliotoxin triggers mitochondria depolarization and DNA fragmentation in HL60S but has no effect on HL60R. The p21 peptide has a more modest effect on mitochondria depolarization in HL60S. Remarkably, the p21 peptide induces a low but significant mitochondria depolarization in HL60R, and induces DNA fragmentation in HL60R more potently than in HL60S.

FIG. 8. Effect of siRNA targeting PCNA on gliotoxin-induced apoptosis in daunorubicin-resistant HL-60 cells. HL60 cells were transfected with control (CT)-siRNA or with PCNA-siRNA (1 mM) using the Amaxa technology (kitV program T019). 24 hours after transfection, HL60 cells were incubated for 4 hours with gliotoxin at 1 or 2 mg/ml. Effect of siRNA on the percentage of apoptotic HL60 cells were measured by mitochondrial depolarization after DiOC₆ labeling. Data are means±SEM of 4 independent experiments. Inhibition of PCNA expression significantly increases gliotoxin-induced apoptosis.

### BRIEF DESCRIPTION OF THE SEQUENCES

SEQ ID No. 1 shows the amino acid sequence of PCNA.
SEQ ID No. 2 shows the amino acid sequence of p21.
SEQ ID No. 3 shows the amino acid sequence of residues 141 to 160 of p21.
SEQ ID No. 4 shows the amino acid sequence of residues 141 to 160 of p21 fused to the RYIRS tag.

### EXAMPLES

### Example 1

### Materials and Methods

### Cell Culture

The K562 and UT7.9 cell lines (Klein et al. Int J Cancer 1976; 18:421-31, Koeffler et al. Blood 1980; 56:344-50, Chretien et al. Blood 1994; 83:1813-21) were cultured in Roswell Park Memorial Institute (RPMI) 1640 supplemented with 10% fetal calf serum (FCS) and antibiotics (penicillin 100 U/ml and streptomycin at 100 μg/ml). Cell lines were maintained at 37° C. in 5% CO₂. The K562 and UT7.9 were resistant to 1 μM doxorubicin and 4 μM imatinib, respectively).

Analysis of Drug Resistance by DIOC2

The assay is based on the efflux of fluorescent P-gp substrate DiOC2 (3-ethyl-2-[3-(3-ethyl-2(3H)-benzoxazolylidene)-1-propenyl]benzoxazolium iodide) in doxorubicin-resistant cells, which can be inhibited by cisplatin. This efflux is absent in doxorubicin-sensitive cells resulting in an accumulation of DIOC2. DIOC2 fluorescence is measured by flow cytometry. Briefly, K562 at 1×10⁶ cellules/ml were treated by cisplatin (2.5 μg/ml) in the presence of DIOC2 (50 ng/ml) for 30 min at 37° C. The cells are analyzed by flow cytometry.

Isolation of Bone Marrow Cells

The bone marrow cells were isolated by gradient percoll as previously described (Cowland et al. J Immunol. Methods 1999; 232:191-200).

PCNA Protein Analysis

Cells were fixed in PBS-3.7% formaldehyde for 20 min in ice and permeabilized with triton (X100) 0.25% for 5 min at room temperature followed by permeabilization with ice-cold methanol for 10 min. Immunolabelling was performed in humid dark chamber using rabbit polyclonal anti-PCNA (Ab5) (PC474, MERCK, Calbiochem, Germany) incubated for 45 min followed by Alexa 555-conjugated rabbit IgG for 30 min (2 mg/ml, Molecular Probes®, Invitrogen). The nuclei were stained by Hoechst at 2 μg/ml for 15 min. Slides were mounted using Fluoprep medium and analyzed by confocal microscopy with a Leica TCS SP5 AOBS imaging microscope X63 and the LAS AF version 1.8 software. The quantification software is Image J.

Analysis of Apoptosis

Externalization of phosphaditylserine was measured by flow cytometry after annexin-V binding (Moriceau et al. J Immunol. 2009; 182:7254-7263).

### Example 2

### The Cellular Localization of PCNA is Linked with Resistance to Apoptosis

The inventors recently observed that neutrophils express high amounts of the Proliferating Cell Nuclear Antigen (PCNA), which belongs to the family of DNA sliding clamps. Moreover, they unexpectedly discovered that, in neutrophils, PCNA localizes exclusively in the cytoplasm, due to a relocalization occurring during myeloid differentiation. Notably, cytosolic PCNA levels changed in parallel with neutrophil survival rate: decreasing during apoptosis, increasing during in vitro or in vivo exposure to the survival factor G-CSF. Moreover, PCNA overexpression rendered neutrophil-differentiated PLB985 myeloid cells significantly more resistant to TRAIL- or gliotoxin-induced apoptosis. These results identified cytoplasmic PCNA as cell cycle independent regulator of neutrophil lifespan. Based on the knowledge that PCNA acts as a protein platform to mediate its biological activities, the inventors identified one of the molecular mechanisms whereby PCNA exerts its anti-apoptotic effect, namely its ability to associate with, and prevent the activation of procaspases.

### Example 3

### Cytoplasmic PCNA is Associated with Drug Resistance in Myelocytic Leukemia Cell Lines

The inventors next examined whether cytoplasmic PCNA could be involved in cell survival in myelocytic leukemia. Indeed, the inventors previously showed that PCNA was localized in the cytoplasm in terminally differentiated cells (positive for MPO and CD35) whilst in earlier stage of differentiation, in promyelocytes (negative for CD35 and positive for MPO) PCNA was localized in the nucleus.

In acute myelocytic leukemia, there is an arrest in the granulocytic differentiation and a proliferation of precursor cells, which are resistant to apoptosis. Interestingly, an increased PCNA expression had been reported in myelocytic leukemia cells but the impact of PCNA localization had not been investigated.

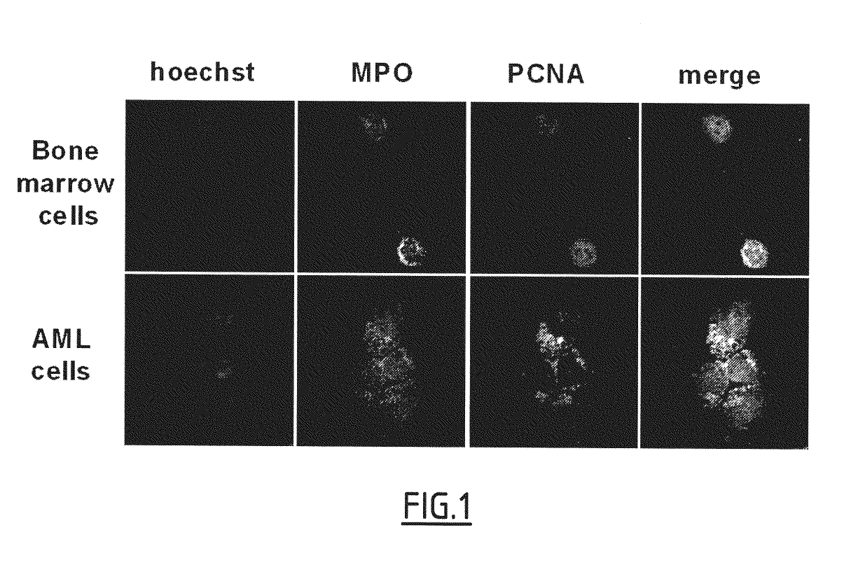

The inventors showed by immunofluorescence that promyelocytes (MPO positive cells) isolated from the bone marrow of a patient with AML showed a strong expression of cytoplasmic PCNA whereas no cytoplasmic PCNA could be detected in bone marrow promyelocytes (MPO positive cells) isolated from a healthy subject (FIG. 1).

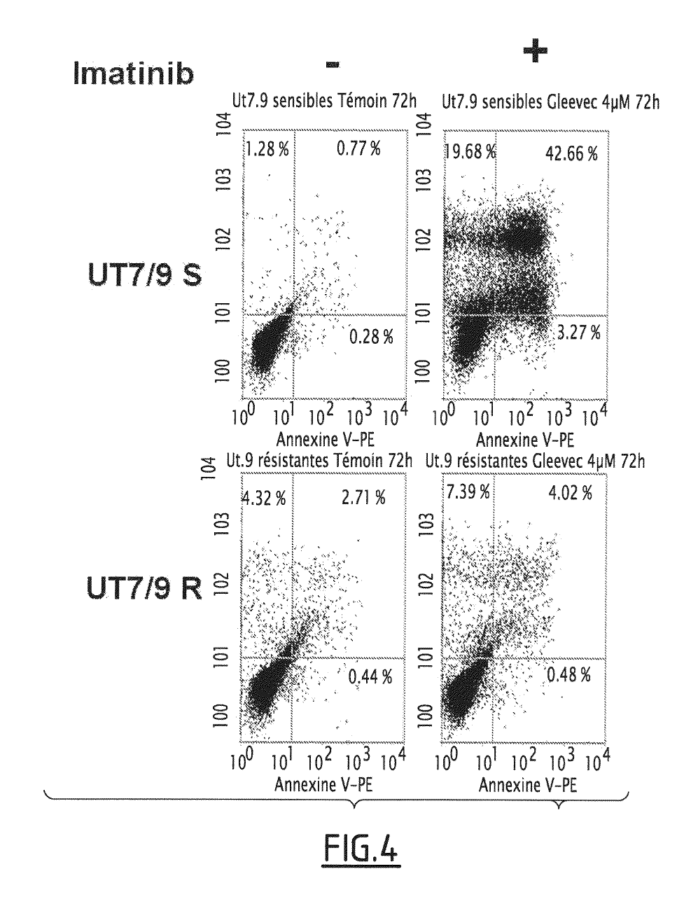

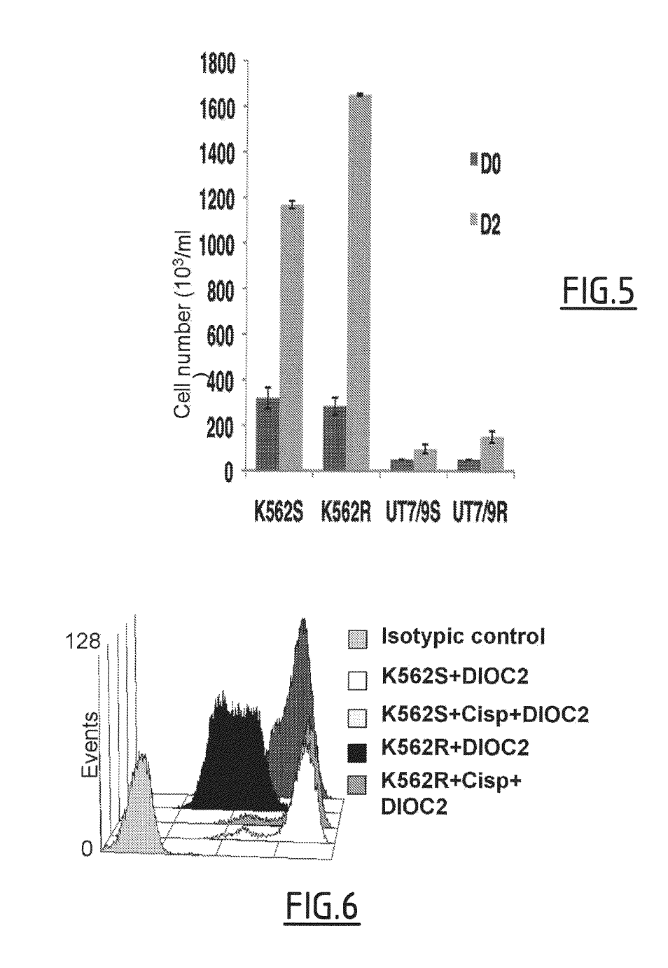

The inventors next evaluated whether cytoplasmic PCNA could be involved in drug resistance in leukemia cell lines. Two myelocytic leukemia cell lines have been studied K562 and UT7.9. These cell lines have a strong expression of BCR-ABL involved in oncogenesis of chronic myelocytic leukemia. This expression is endogenous in K562 cells and induces by retroviral gene transfer in UT7 cells to generate clone UT7.9. Appearance of resistance of clones was observed by imatinib mesilate or anthracyclins (like doxorubicin) treatments and the mechanisms of this drug resistance were not completely understood. The inventors showed a drug resistance of K562 and UT7.9 cell line, both observed as an increased proliferation (FIG. 5) and as an increased cell survival (FIGS. 4 and 6), compared to the respective sensitive cell lines.

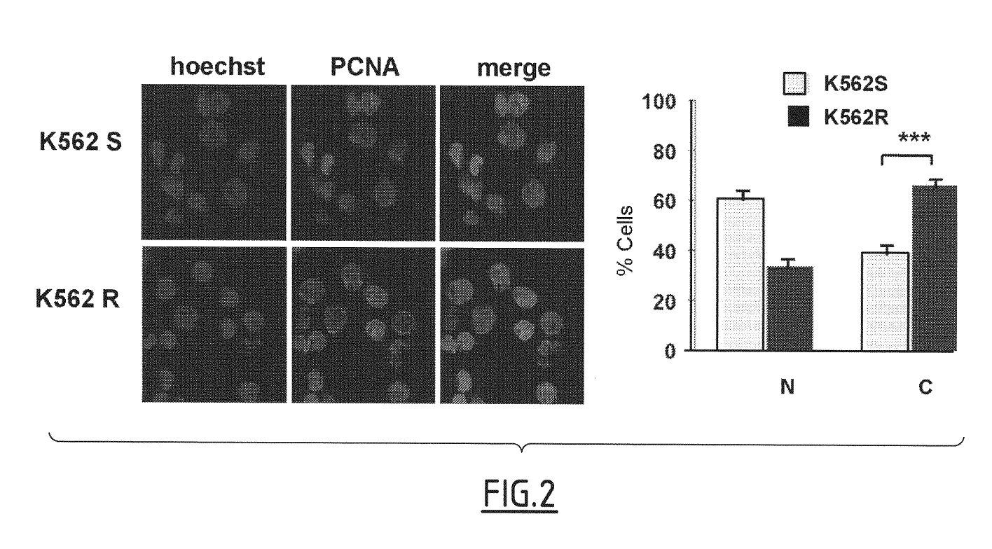

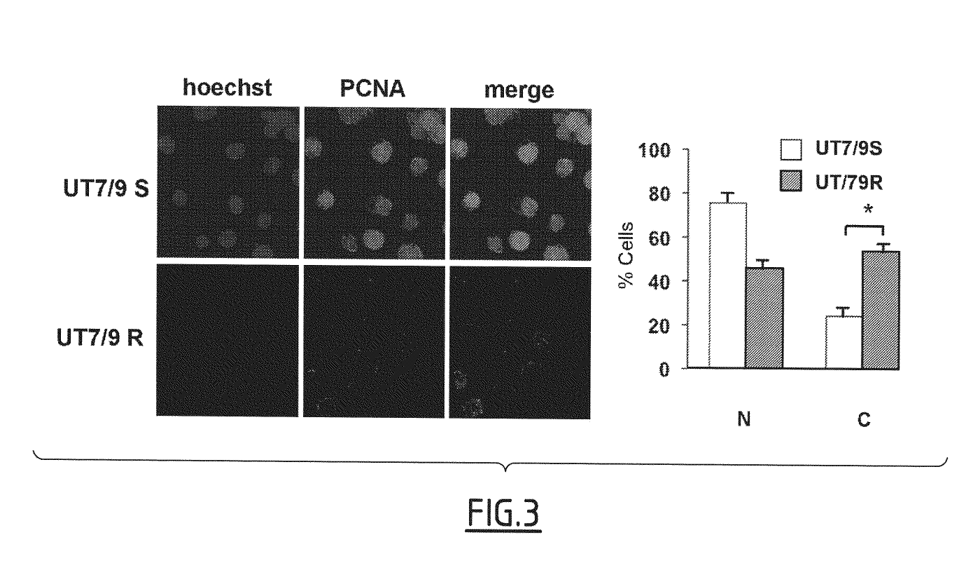

Immunofluorescence analysis showed that PCNA was cytoplasmic in 66% of doxorubicin-resistant compared to 39% of sensitive K562 cells (FIG. 2). Similar findings were found in UT7/9 cells, with 54% and 24% of cells presenting cytoplasmic PCNA in imatinib mesilate-resistant and sensitive cells, respectively (FIG. 3).

These findings indicate that cytoplasmic PCNA was associated with drug resistance in myelocytic leukemia cell line.

### Example 4

### PCNA Localization Allows Discriminating Between Two Groups of Patients Suffering from AML

CD34+ myeloid precursor cells isolated from healthy donors were used as control. In these cells, 30% of the cells express PCNA in the cytoplasm and 70% of the cells express PCNA exclusively in the nucleus. In order to standardize the quantification, fluorescence analyses were performed using a spinning disk microscope. This technique allows measuring different parameters including the number of cells by field, the nucleus area, PCNA fluorescence area in the nucleus, PCNA fluorescence area in the whole cell. This technique allows validating the distribution of PCNA in the CD34+ control cells: 30% of the PCNA fluorescence is in the cytoplasm and 70% of the PCNA fluorescence is in the nucleus.

The inventors then studied cells isolated from six patients suffering from acute myeloid leukaemia (ALM). These patients are treated in the haematology service headed by Pr. Didier Bouscary in the Cochin Hospital.

PCNA immuno-staining in the cells isolated from the leukaemia patients blood shows a high heterogeneity in PCNA distribution. This heterogeneity could be in relation with the clinical heterogeneity observed in ALM. Thus, two groups of patients can be discriminated: in the first group (two patients), PCNA is mainly expressed in the nucleus (70%), whereas in the second group (four patients), PCNA is mainly expressed in the cytoplasm (60%).

PCNA can thus be differently localized according to the group of patients. Therefore, leukemic patients can be classified according to the proportion or their leukemic cells expressing cytoplasmic PCNA, in order to predict their responsiveness to a chemotherapeutic drug.

### Example 5

### Knocking Down PCNA Expression by siRNA Sensitizes Daunorubicin-Resistant HL-60 Cells to Gliotoxin-Induced Apoptosis

SiRNA were used to knock down PCNA expression in HL-60 cells, which are both resistant to daunorubicin and to gliotoxin-induced apoptosis. HL60 cells were transfected with control (CT)-siRNA or with PCNA-siRNA (1 mM) using the Amaxa technology (kitV program T019). 24 hours after transfection, HL60 cells were incubated for 4 hours with gliotoxin at 1 or 2 mg/ml. Effect of siRNA on the percentage of apoptotic HL60 cells were measured by mitochondrial depolarization after DiOC₆ labeling.

The results show that inhibition of PCNA synthesis in HL60R cells significantly increases gliotoxin-induced apoptosis (FIG. 8). These data confirm that PCNA is a potential target in anti-leukemic treatment.

### Example 6

### The p21 Peptide Restores Apoptosis in Daunorubicin-Resistant HL60 Cells

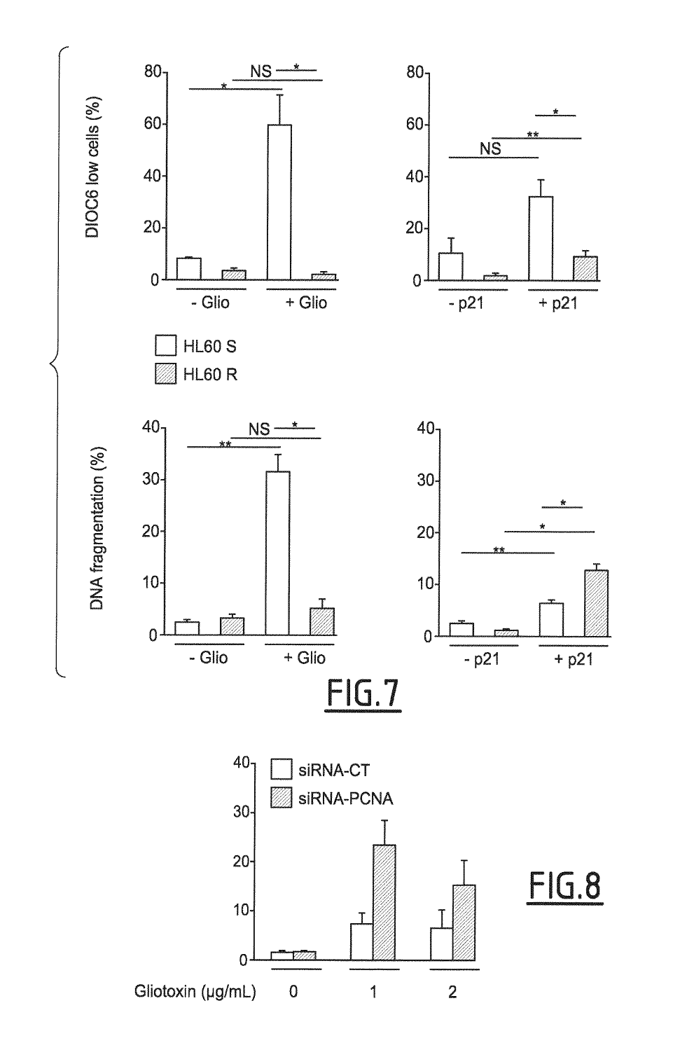

Apoptosis in HL-60 cells sensitive to daunorubicin (HL60S) was compared to apoptosis in HL-60 cells resistant to daunorubicin (HL60R). Apoptosis was induced either by gliotoxin that triggers apoptosis by targeting the mitochondria or the p21 peptide. The p21 peptide corresponds to residues 141 to 160 of p21 fused to the cell penetrating peptide RYIRS. HL60S and HL60R were incubated overnight with the p21 peptide (50 mM) or with gliotoxin (1 mg/ml). After a 15-hour incubation, the effect of the p21 peptide on the percentage of apoptotic HL60 cells was measured by mitochondrial depolarization after DiOC₆ labeling and by DNA fragmentation after propidium iodide labeling (FIG. 7).

Gliotoxin triggers mitochondria depolarization in HL60S (60%) but has no effect on HL60R, thus confirming that these latter cells are resistant to apoptosis triggered via the mitochondria pathway. In contrast, the p21 peptide has a more modest effect on mitochondria depolarization in HL60S than gliotoxin. Remarkably, the p21 peptide induced a low but significant mitochondria depolarization in HL60R.

Using DNA fragmentation as a read out of apoptosis, the inventors confirmed that gliotoxin induces apoptosis in HL60S but not in HL60 R cells. Interestingly, the p21 peptide appears to have a more pronounced effect in HL60R than on HL60S, inducing DNA fragmentation in HL60R more potently than in HL60S. These data confirm that the p21 peptide may be used in combination with anti-leukemic treatment to potentiate apoptosis.

1. A method for predicting the responsiveness of an individual suffering from leukemia to a chemotherapeutic drug, said method comprising determining the proportion of leukemic cells expressing cytoplasmic PCNA in a biological sample of the individual.

2. The method of claim 1, wherein a proportion of leukemic cells expressing cytoplasmic PCNA higher than a predetermined threshold is indicative that the individual is likely not to respond to the chemotherapeutic drug.

3. The method of claim 1, wherein a proportion of leukemic cells expressing cytoplasmic PCNA of at least 50% is indicative that the individual is likely not to respond to the chemotherapeutic drug.

4. The method of claim 1, wherein the chemotherapeutic drug is selected from the group consisting of alkaloids, alkylating agents, antimetabolites, antibiotics, tyrosine kinase inhibitors, topoisomerase inhibitors, monoclonal antibodies, biological response modifiers and corticosteroids.

5. The method of claim 1, wherein the chemotherapeutic drug is a tyrosine kinase inhibitor or a topoisomerase inhibitor.

6. The method of claim 1, wherein the chemotherapeutic drug is imatinib mesilate or doxorubicin.

7. The method of claim 1, wherein the leukemia is a myelocytic leukemia.

8. The method of claim 1, wherein the leukemia is selected from the group consisting of acute myelocytic leukemia and chronic myelocytic leukemia.

9. The method of claim 1, further comprising the step of designing a treatment regimen.

10. The method of claim 1, wherein said step of determining the proportion of leukemic cells expressing cytoplasmic PCNA in a biological sample of the individual is repeated at least at two different points in time.

11. A chemotherapeutic drug for use for the treatment of an individual suffering from leukemia, said individual having a proportion of leukemic cells expressing cytoplasmic PCNA in a biological sample lower than a predetermined threshold.

12. The chemotherapeutic drug of claim 11, wherein the individual has a proportion of leukemic cells expressing cytoplasmic DNA of at most 50%.

13. The chemotherapeutic drug of claim 11, wherein the chemotherapeutic drug is imatinib mesilate or doxorubicin.

14. The chemotherapeutic drug of claim 11, wherein the leukemia is a myelocytic leukemia.

15. A combination of a chemotherapeutic drug and an antagonist of cytoplasmic PCNA for use for the treatment of an individual suffering from leukemia, said individual having a proportion of leukemic cells expressing cytoplasmic PCNA in a biological sample higher than a predetermined threshold.

16. The combination for use according to claim 15, wherein the individual has a proportion of leukemic cells expressing cytoplasmic DNA of at least 50%.

17. The combination for use according to claim 15, wherein said antagonist of cytoplasmic PCNA is selected from the group consisting of a siRNA targeting PCNA, a shRNa targeting PCNA, and a fragment of p21 comprising residues 141 to 160 of SEQ ID NO: 2.

18. An in vitro method for selecting a patient suffering from leukemia suitable to be treated with a therapy comprising a combination of a chemotherapeutic drug and an antagonist of cytoplasmic PCNA, said method comprising the steps of:
a) providing or obtaining a biological sample comprising leukemic cells;
b) determining the proportion of leukemic cells expressing cytoplasmic PCNA in said biological sample; and
c) selecting the patient if it has a proportion of leukemic cells expressing cytoplasmic PCNA higher than a predetermined threshold.

19. The method of claim 18, wherein step (c) comprises selecting the patient if said patient has a proportion of leukemic cells expressing cytoplasmic PCNA higher than 50%.

20. The method according to claim 18, wherein said antagonist of cytoplasmic PCNA is selected from the group consisting of a siRNA targeting PCNA, a shRNa targeting PCNA, and a fragment of p21 comprising residues 141 to 160 of SEQ ID NO: 2.
